(19) United States
(12) Patent Application Publication
(10) Pub. No.: US 2015/0059758 A1
(43) Pub. Date: Mar. 5, 2015

(54) SELECTABLE EXHAUST PORT ASSEMBLY

(71) Applicant: KONINKLIJKE PHILIPS N.V., EINDHOVEN (NL)

(72) Inventor: Jerome Matila, JR., Apollo, PA (US)

(21) Appl. No.: 14/387,580

(22) PCT Filed: Mar. 5, 2013
(86) PCT No.: PCT/IB2013/051731

§ 371 Date: Sep. 24, 2014

Related U.S. Application Data
(60) Provisional application No. 61/615,600, filed on Mar. 26, 2012.

(51) Int. Cl. A61M 16/08; A61M 16/06; A61M 16/00

(52) U.S. Cl. CPC A61M 16/0816; A61M 16/0057; A61M 16/06; A61M 16/0883; A61M 2206/20
USPC 128/205.25; 128/204.18

(57) ABSTRACT

An exhaust port assembly (10) for use in a system for delivering a flow of gas from a pressure generating device (4) to the airway of a patient includes a first member structured to be in communication with the flow of gas and a second member moveably coupled to the first member. The first member and the second member define a cross-sectional area of an exhaust port which is structured to allow the passage therethrough of exhaust gases (12) from the flow of gas. The second member is moveable among a first position in which the exhaust port has a first cross-sectional area and a second position in which the exhaust port has a second cross-sectional area different than the first cross-sectional area.

### CROSS-REFERENCE TO RELATED APPLICATIONS

This patent application claims the priority benefit under 35 U.S.C. §119(e) of U.S. Provisional Application No. 61/615,600, filed on Mar. 26, 2012, the contents of which are herein incorporated by reference.

### BACKGROUND OF THE INVENTION

1. Field of the Invention

The present invention relates to the control of flow patterns of fluids, such as gases, and, more particularly, to an adjustable exhaust port assembly that may be employed in, for example, a respiratory patient interface system. The invention also relates to systems incorporating adjustable exhaust port assemblies.

2. Description of the Related Art

It is well known to treat a patient with a non-invasive positive pressure support therapy, in which a flow of breathing gas is delivered to the airway of a patient at a pressure greater than the ambient atmospheric pressure. For example, it is known to use a continuous positive airway pressure (CPAP) device to supply a constant positive pressure to the airway of a patient throughout the patient's respiratory cycle to treat obstructive sleep apnea (OSA), as well as other cardio-pulmonary disorders, such as congestive heart failure (CHF) and cheynes-stokes respiration (CSR). Examples of such CPAP devices include the REMstar® family of CPAP devices manufactured by Philips Respironics, Inc. of Murrysville, Pa.

A “bi-level” non-invasive positive pressure therapy, in which the pressure of gas delivered to the patient varies with the patient's breathing cycle, is also known. Such a bi-level pressure support system provides an inspiratory positive airway pressure (IPAP) that is greater than an expiratory positive airway pressure (EPAP). IPAP refers to the pressure of the flow of gas delivered to the patient's airway during the inspiratory phase; whereas EPAP refers to the pressure of the flow of gas delivered to the patient's airway during the expiratory phase. Such a bi-level mode of pressure support is provided by the BiPAP® family of devices manufactured and distributed by Phillips Respironics, Inc.

Auto-titration positive pressure therapy is also known. With auto-titration positive pressure therapy, the pressure of the flow of breathing gas provided to the patient changes based on the detected conditions of the patient, such as whether the patient is snoring or experiencing an apnea, hypopnea, or upper airway resistance. An example of a device that adjusts the pressure delivered to the patient based on whether or not the patient is snoring is the REMStar Auto family of devices manufactured and distributed by Respironics, Inc.

Other modes of providing positive pressure support to a patient are known.

For example, a proportional assist ventilation (PAV®) mode of pressure support provides a positive pressure therapy in which the pressure of gas delivered to the patient varies with the patient's breathing effort to increase the comfort to the patient. Proportional positive airway pressure (PPAP) devices deliver breathing gas to the patient based on the flow generated by the patient.

For purposes of the present invention, the phrases “pressure support device”, “pressure generating device”, and/or “pressure generator” (used interchangeably herein) refer to any medical device adapted for delivering a flow of breathing gas to the airway of a patient, including a ventilator, CPAP, PAV, PPAP, or bi-level pressure support device. The phrases “pressure support system” and/or “positive pressure support system” (used interchangeably herein) include any arrangement or method employing a pressure support device and adapted for delivering a flow of breathing gas to the airway of a patient.

In a conventional pressure support system, a flexible conduit couples the pressure support device to a patient interface device. The flexible conduit forms part of what is typically referred to as a “patient circuit”, which carries the flow of breathing gas from the pressure support device to patient interface device. The patient interface device connects the patient circuit with the airway of the patient so that the flow of breathing gas is delivered to the patient's airway. Examples of patient interface devices include a nasal mask, nasal and oral mask, full face mask, nasal cannula, oral mouthpiece, tracheal tube, endotracheal tube, or hood.

In a non-invasive pressure support system, i.e., a system that remains outside the patient, a single-limb patient circuit is typically used to communicate the flow of breathing gas to the airway of the patient. An exhaust port (also referred to as an exhalation vent, exhalation port, and/or exhaust vent) is provided in the patient circuit and/or the patient interface device to allow exhaust gas, such as the exhaled gas from the patient, to vent to atmosphere.

A variety of exhalation ports are known for venting gas from a single-limb patient circuit. For example, U.S. Pat. No. Re. 35,339 to Rappoport discloses a CPAP pressure support system wherein a few exhaust ports are provided directly on the patient interface device, i.e., in the wall of the mask. Such exhaust ports are of fixed size which, while optimum for gas flows at particular pressures, are less than ideal for other situations.

Current pressure generating devices used for treating sleep apnea can supply patient delivery pressures ranging from 4 to 20 cmH₂O in 1/2 cmH₂O increments. The volume of inspired and expired air in a patient is determined by an individuals' physiology, and the same amount of air delivered to the patient must be exhausted to the atmosphere to eliminate CO₂ rebreathing within the circuit. Fixed size exhaust ports provide different flow rates at different pressures, thereby exhausting a low volume of air at low pressures and higher amounts at high pressures. This discrepancy may cause inadequate venting at low pressure or excess venting at high pressure. Present exhalation ports compromise between the two extremes to provide safe leak rates at both extremes, but they are not designed for a specific leak rate.

### SUMMARY OF THE INVENTION

In one embodiment of the invention, an exhaust port assembly for use in a system for delivering a flow of gas from a pressure generating device to the airway of a patient is provided. The exhaust port assembly comprises: a first member structured to be in communication with the flow of gas and a second member moveably coupled to the first member. The first and second members define a cross-sectional area of an exhaust port which is structured to allow the passage therethrough of exhaust gases from the flow of gas. The second member is moveable among a first position in which the exhaust port has a first cross-sectional area and a second position in which the exhaust port has a second cross-sectional area different than the first cross-sectional area.

The first member may comprise a portion of a patient interface or a portion of a patient circuit and may include a first aperture of predetermined cross-sectional area formed therein. The second member may comprise a dial-like member having a plurality of second apertures of varying cross-sectional areas formed therein, the second member being rotatably coupled to the first member in a manner such that each of the second apertures may be selectably aligned with the first aperture. The cross-sectional area of the exhaust port may be defined by the one of the plurality of second apertures aligned with the first aperture.

The first member may comprise at least a portion of a first tubular member structured to conduct the flow of gas therethrough and the second member may comprise at least a portion of a second tubular member disposed about the first member. The first tubular member may disposed about a longitudinal axis and the second member may be slidable axially along the longitudinal axis.

The first member may comprise an aperture having a length disposed parallel to the longitudinal axis and a width disposed perpendicular to the longitudinal axis, the width varying along the length thereof and the second member may be disposed to selectively block a portion of the aperture. The cross-sectional area of the exhaust port may be defined by a portion of the aperture not blocked by the second member.

The first tubular member may be disposed about a longitudinal axis and the second member may be rotatable about the longitudinal axis. The first member may comprise a first aperture having a length disposed perpendicular to the longitudinal axis and a width disposed parallel to the longitudinal axis, the width varying along the length thereof. The second member may comprise a second aperture having a length disposed along the longitudinal axis, the length being equal to or greater than the width of the first aperture. The cross-sectional area of the exhaust port may be defined by a portion of the first aperture aligned with the second aperture.

The first member may comprise a first aperture of predetermined cross-sectional area formed therein. The second member may comprise a plurality of second apertures of varying cross-sectional areas equal to, or smaller than, the cross sectional area of the first aperture, formed therein. The second member may be rotatably coupled to the first member in a manner such that each of the second apertures may be selectably aligned with the first aperture. The cross-sectional area of the exhaust port may be defined by the one of the plurality of second apertures aligned with the first aperture.

The first member may comprise a plurality of first apertures of varying cross-sectional areas formed therein. The second member may comprise a second aperture of predetermined cross-sectional area formed therein, the predetermined cross-sectional area of the second aperture being equal to, or larger than any of the cross-sectional areas of the plurality of first apertures. The second member may be rotatably coupled to the first member in a manner such that the second aperture may be selectably aligned with each of the plurality of first apertures. The cross-sectional area of the exhaust port may be defined by the one of the plurality of first apertures to which the second aperture is aligned.

The first member may comprise an aperture having a cross-sectional area and the second member may comprise a plurality of second members slidably disposed about the periphery of the aperture. The cross-sectional area of the exhaust port may be defined by a portion of the aperture not blocked by the plurality of second members.

As another aspect of the invention, a system for delivering a flow of treatment gas to the airway of a patient is provided. The system comprises a pressure generating device, a patient interface , a patient circuit structured to deliver the flow of treatment gas from the pressure generating device to the patient interface, and an exhaust port assembly as previously discussed.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a schematic diagram of a known pressure support system adapted to provide a regimen of respiratory therapy to a patient;

FIGS. 2, 3, 4A, 5A, 6A, 7A and 8A show example embodiments of exhaust port assemblies according to embodiments of the present invention; and

FIGS. 4B, 5B, 6B, 7B and 8B, respectively, show the exhaust port assemblies of FIGS. 4A, 5A, 6A, 7A and 8A disposed in second positions in which the exhaust port size has been selectively varied.

### DETAILED DESCRIPTION OF THE EXEMPLARY EMBODIMENTS

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As employed, herein, the statement that two or more parts or components are “coupled” together shall mean that the parts are joined or operate together either directly or through one or more intermediate parts or components.

As employed herein, the statement that two or more parts or components “engage” one another shall mean that the parts exert a force against one another either directly or through one or more intermediate parts or components.

As employed herein, the term “number” shall mean one or an integer greater than one (i.e., a plurality) and the singular form of “a”, “an”, and “the” include plural referents unless the context clearly indicates otherwise. As employed herein, the term “define” shall mean that one or more elements form the boundaries of a particular element.

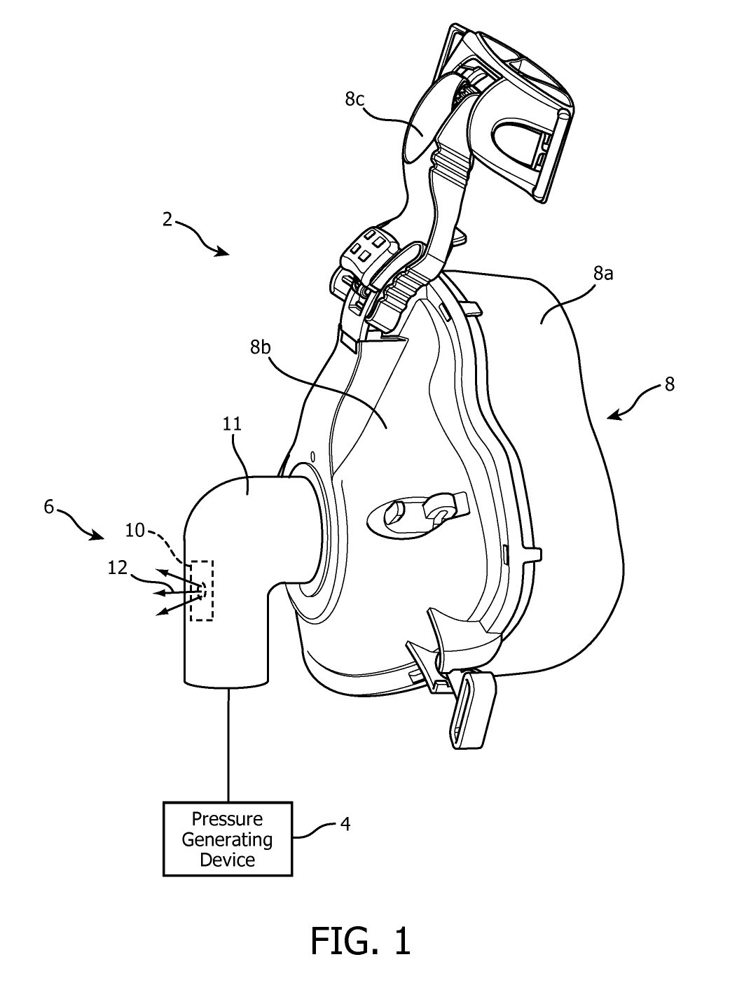

A system 2 adapted to provide a regimen of respiratory therapy to a patient is generally shown in FIG. 1. System 2 includes a pressure generating device 4, a patient circuit 6, a patient interface device 8, and an exhaust port assembly 10 (shown schematically in dashed line) included on an elbow 11 along patient circuit 6. Although system 2 is discussed as including pressure generating device 4, patient circuit 6, and patient interface device 8, it is contemplated that other systems may be employed while remaining within the scope of the present invention. For example, and without limitation, a system in which the pressure generating device is coupled to a patient interface device having an integrated exhaust port assembly 10 is contemplated.

Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices.

Patient circuit 6 is structured to communicate the flow of breathing gas from pressure generating device 4 to patient interface device 8. Typically, patient circuit 6 includes a conduit or tube which couples pressure generating device 4 and patient interface device 8. In the current embodiment, conduit 6 includes an elbow 11 coupled to the interface device 8 which includes exhaust port assembly 10 which allows for the venting of exhaust gases 12 therefrom.

Patient interface device 8 is typically a nasal or nasal/oral mask structured to be placed on and/or over the face of a patient. Any type of patient interface device 8, however, which facilitates the delivery of the flow of breathing gas to, and the removal of a flow of exhalation gas from, the airway of such a patient may be used while remaining within the scope of the present invention. In the example shown in FIG. 1, patient interface device 8 includes cushion 8a, rigid shell 8b, and forehead support 8c. Straps (not shown) may be attached to shell 8b and forehead support 8c to secure patient interface device 8 to the patient's head.

An opening in shell 8b, to which exhaust elbow 11 is coupled, allows the flow of breathing gas from pressure generating device 4 to be communicated to an interior space defined by shell 8b and cushion 8a, and then, to the airway of a patient. The opening in shell 8b also allows the flow of exhalation gas (from the airway of such a patient) to be communicated to elbow 11 and exhaust port assembly 10 in the current embodiment. Although illustrated in a separate elbow component 11 in FIG. 1, it is contemplated that exhaust port assembly 10 may be incorporated into, for example and without limitation, patient interface 8 and/or different variations of patient circuit 6 while remaining within the scope of the present invention.

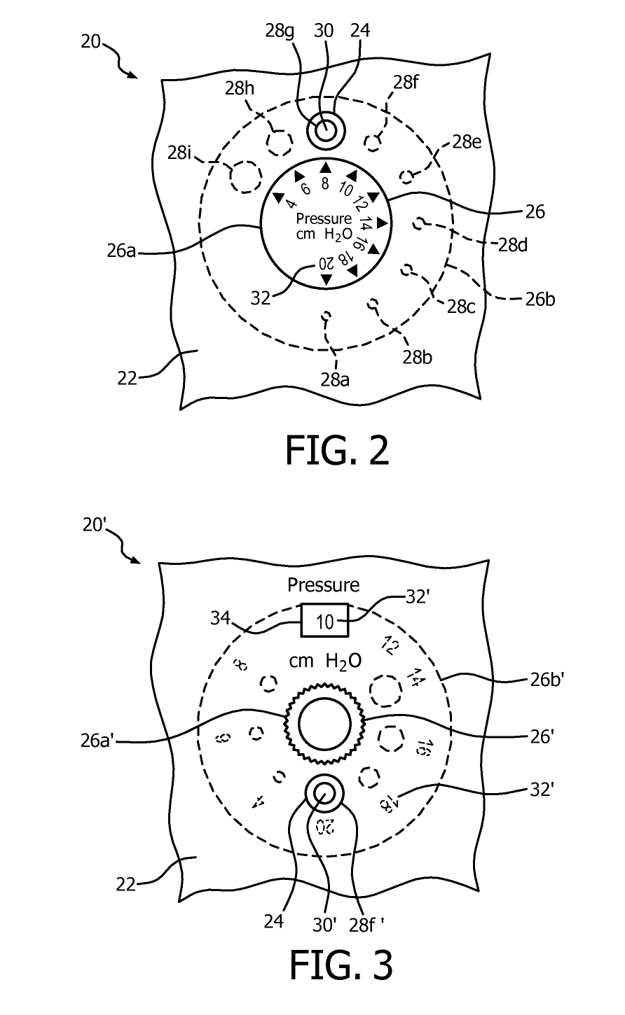

Having thus described the general components of system 2, detailed descriptions of example exhaust port assemblies in accordance with the present invention will now be described in reference to FIGS. 2, 3, 4A and 4B, 5A and 5B, 6A and 6B, 7A and 7B, and 8A and 8B.

Referring to FIG. 2, an example embodiment of an exhaust port assembly 20 is shown disposed in a portion 22 of a patient interface device, such as patient interface device 8 previously discussed. Portion 22 forms a first member of exhaust port assembly 20 as portion 22 includes a first aperture 24 of predetermined cross-sectional area formed therein which is structured to permit the flow of exhaust gases therethrough. Although shown as being generally circular, it is to be appreciated that first aperture 24 may be of other shape without varying from the scope of the present invention. Continuing to refer to FIG. 2, exhaust port assembly 20 further includes a second member 26 rotatably coupled to portion 22. Second member 26 is formed generally as a dial-like member and includes a first portion 26a extending generally from portion 22, and a second portion 26b disposed on the opposite (patient facing) side of portion 22. Second portion 26b includes a plurality of second apertures 28a-28i of varying cross-sectional areas formed therein.

In use, exhaust port assembly 20 allows for the flow of exhaust gases therethrough to be selectively adjusted by adjusting the cross-sectional area of the actual exhaust port 30 as defined by the first member (portion 22) and the second member (dial-like member 26). In the embodiment shown in FIG. 2, the exhaust port 30 has a cross-sectional area equal to that of the second aperture 28g, as second aperture 28 is shown aligned with first aperture 24. The cross-sectional area of exhaust port 30 may be selectively varied by rotating second member 26 with respect to portion 22 so that another one of second apertures 28a-28i is generally aligned with first aperture 24.

In order to inhibit the potential undesired escape of gases through anywhere other than through the selected one of second apertures 28a-28i and first aperture 24, second portion 26b of second member 26 is generally sealed with the patient side (not numbered) of portion 22. As shown in the example embodiment of FIG. 2, first portion 26a of second member 26 may be provided with indicia 32 which provide a suggestion of the exhaust port 30 to be used with particular operating pressures.

FIG. 3 shows another example of an exhaust port assembly 20′ similar to exhaust port assembly 20 previously discussed. Exhaust port assembly 20′ operates in a similar manner as exhaust port assembly 20, however exhaust port assembly 20′ instead provides indicia 32′ on second portion 26b′ of second member 26. Such indicia 32′ corresponding to the selected second aperture (second aperture 28f is shown selected in the example of FIG. 3) is viewable by a user through a viewing aperture 34 provided in portion 22. Viewing aperture 34 may be provided as a cut out portion or as a clear portion of portion 22 without varying from the scope of the present invention.

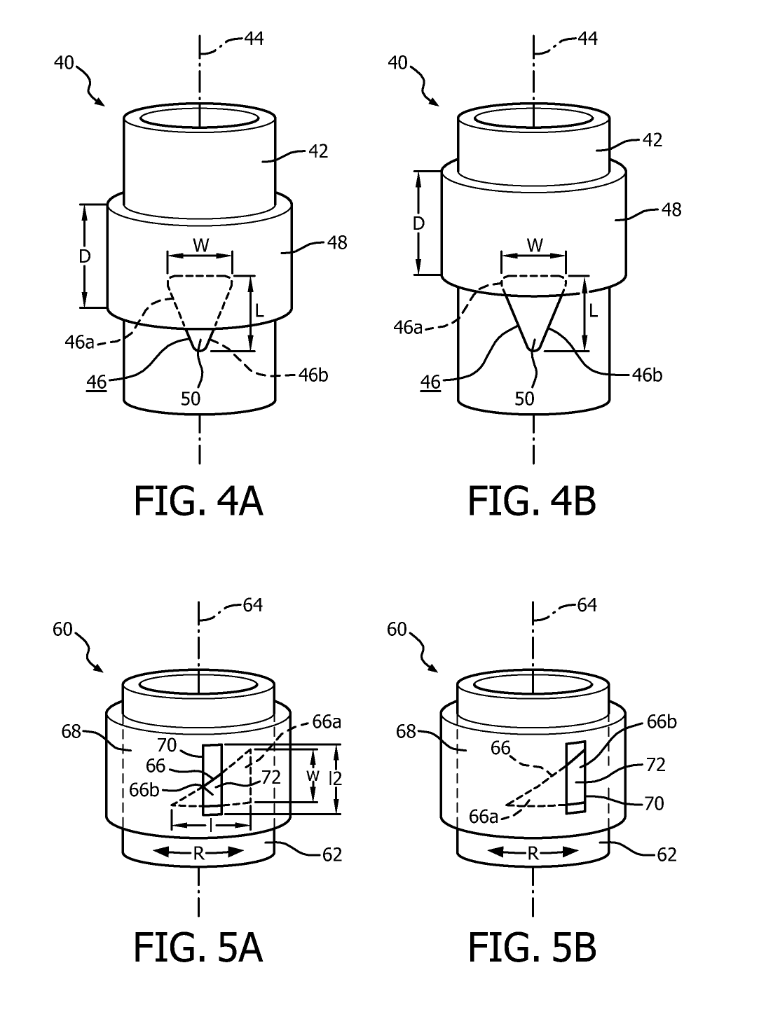

FIGS. 4A and 4B show another example of an exhaust port assembly 40 according to another embodiment of the present invention shown in two different positions. Exhaust port assembly 40 includes a first member 42 disposed generally about a longitudinal axis 44. First member 42 is formed as a generally tubular member structured to conduct the flow of gas therethrough, such as a portion of, or a coupling connected with conduit 6 of FIG. 1. First member 42 includes an aperture 46 (shown partially in hidden line) having a length L disposed parallel to longitudinal axis 44 and a width W disposed perpendicular to longitudinal axis 44. The width W varying along length L.

Continuing to refer to FIGS. 4A and 4B, exhaust port assembly 40 also includes a second member 48 formed generally as a tubular member disposed about first member 42 such that second member 48 is slidable (as shown by arrow D) relative to first member 42 along longitudinal axis 44 such that second member 48 may selectively block a first portion 46a of aperture 46, while leaving a second portion 46b of aperture 46 open to the surrounding environment. Second portion 46b thus defines an exhaust port 50 of exhaust port assembly 40, through which gases may exit first member 42. As shown in FIG. 4A, second member 48 is positioned in a first position covering a large area (first portion 46a) of aperture 46, thus leaving a small area (second portion 46b) uncovered thus defining an exhaust port 50 of relatively small cross-sectional area. In contrast, FIG. 4B shows second member 48 positioned in a second position in which a smaller area (first portion 46a) of aperture 46 is covered, thus leaving a larger area (second portion 46b) of aperture open to form exhaust port 50. Although shown in two particular positions, it is to be appreciated that second member 48 may be positioned in any number of positions from fully covering aperture 46 (and thus not allowing any flow therethrough) to not covering and portion of aperture 46 (and thus not obstructing any flow) without varying from the scope of the present invention.

FIGS. 5A and 5B show yet another example of an exhaust port assembly 60 according to another embodiment of the present invention shown in two different positions. Exhaust port assembly 60 includes a first member 62 disposed generally about a longitudinal axis 64. First member 62 is formed as a generally tubular member structured to conduct the flow of gas therethrough, such as a portion of, or a coupling connected with conduit 6 of FIG. 1. First member 62 includes a first aperture 66 (shown partially in hidden line) having a length l disposed perpendicular to longitudinal axis 64 and a width w disposed parallel to longitudinal axis 64. The width w varying along length 1.

Continuing to refer to FIGS. 5A and 5B, exhaust port assembly 60 also includes a second member 68 having a second aperture 70 formed therein, second aperture 70 having a length l₂, which is equal to or greater than the width w of the first aperture, disposed along (parallel to) longitudinal axis 64. Second member 68 is formed generally as a tubular member disposed about first member 62 such that second member 68 is rotatable (as shown by arrow R) relative to first member 62 about longitudinal axis 64 such that second aperture 70 of second member 68 may selectively block a one or more first portions 66a of first aperture 66, while selectively exposing a second portion 66b of aperture 66 open to the surrounding environment. Second portion 66b thus defines an exhaust port 72 of exhaust port assembly 60 through which gases may exit first member 62. As shown in FIG. 5A, second member 68, and thus second aperture 70 is positioned in a first position in which only a small area (second portion 66b) of aperture 66 is exposed, thus defining an exhaust port 72 of relatively small cross-sectional area. In contrast, FIG. 5B shows second member 68, and thus second aperture 70 positioned in a second position in which a larger area (second portion 66b) of aperture 66 is exposed, thus defining an exhaust port 72 of relatively large cross-sectional area. Although shown in two particular positions, it is to be appreciated that second member 68, and thus second aperture 70, may be positioned in any number of positions from fully not exposing any of first aperture 66 (and thus not allowing any flow therethrough) to exposing a relatively large portion of aperture 66 (and thus allowing a relatively large flow) without varying from the scope of the present invention.

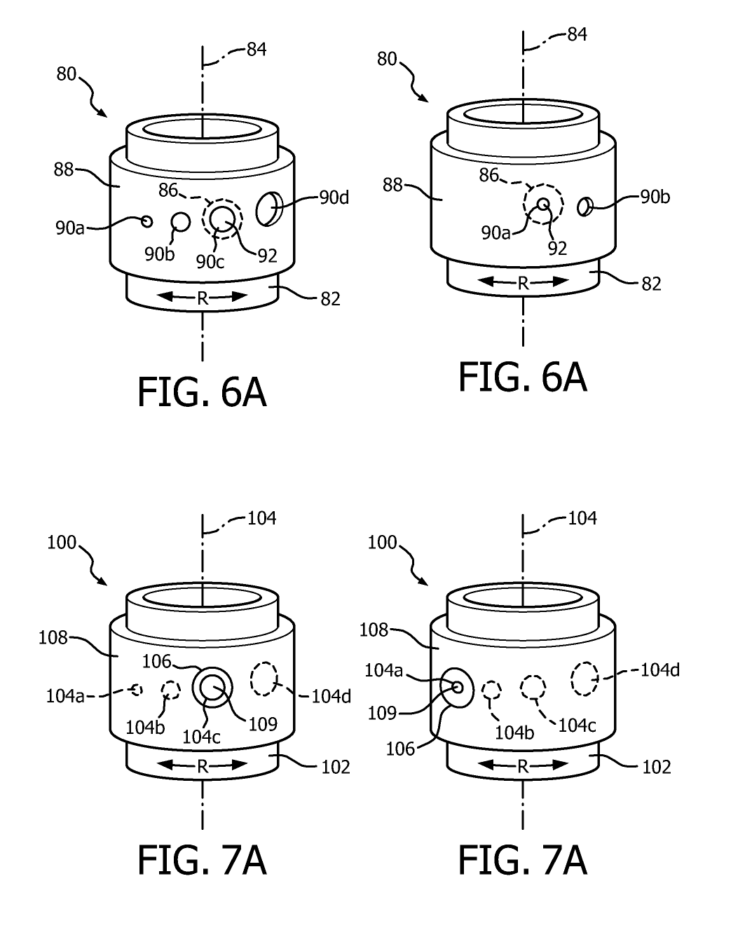

FIGS. 6A and 6B, as well as FIGS. 7A and 7B, show further embodiments of exhaust port assemblies according to the present invention which utilize a combination of some of the concepts previously discussed.

Referring to FIGS. 6A and 6B, exhaust port assembly 80 includes a first member 82 disposed generally about a longitudinal axis 84. First member 82 is formed as a generally tubular member structured to conduct the flow of gas therethrough, such as a portion of, or a coupling connected with conduit 6 of FIG. 1. First member 82 includes a first aperture 86 (shown in hidden line) having a generally circular cross-section (although other shapes may be employed without varying from the scope of the present invention). Exhaust port assembly 80 further includes a second member 88 having a plurality of second apertures 90a-90d of varying size formed therein. Second member 88 is formed generally as a tubular member disposed about first member 82 such that second member 88 is rotatable (as shown by arrow R) relative to first member 82 about longitudinal axis 84 such that a selected one of the plurality of second apertures 90a-90d of second member 88 may be generally aligned with first aperture 86, thus defining an exhaust port 92 through which gases may exit first member 82.

Like the embodiments described in conjunction with FIGS. 2 and 3, exhaust port assembly 80 allows for the flow of exhaust gases therethrough to be selectively adjusted by adjusting the cross-sectional area of the actual exhaust port 92 as defined by first aperture 86 of first member 82 and the selected second aperture (90c in FIG. 6A and 90a of FIG. 6B) of second member 88. When positioned as shown in FIG. 6A, exhaust port 92 has a cross-sectional area equal to that of the second aperture 90c, as second aperture 90c is shown aligned with first aperture 86. In contrast, when positioned as shown in FIG. 6B, exhaust port 92 has a cross-sectional area equal to that of the second aperture 90a, as second aperture 90a is shown aligned with first aperture 86. In order to inhibit the potential undesired escape of gases through anywhere other than through the selected one of second apertures 90a-90d, second member 88 is generally sealed with the outer portion (not numbered) of first portion 82.

The example exhaust port assembly 100 of FIGS. 7A and 7B generally operates in the same manner as exhaust port assembly 80 previously described, however, exhaust port assembly 100 utilizes a generally opposite arrangement of apertures. More particularly, exhaust port assembly 100 utilizes a first member 102 having a plurality of first apertures 104a-104d of varying size formed therein, and only a single second aperture 106 formed in a second member 108 rotatably (along arrow R) coupled to first member 102. Through such arrangement, an exhaust port 109 is defined, which in FIG. 7A is defined by first aperture 104c, and by first aperture 104a in FIG. 7B.

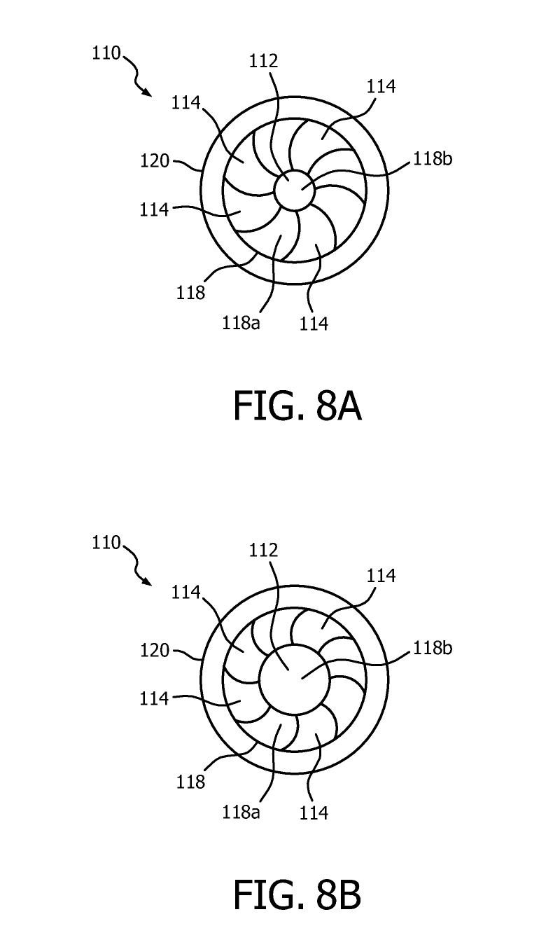

FIGS. 8A and 8B show an example of an exhaust port assembly 110 which employs an iris-type mechanism for varying the size of an exhaust port 112. The iris-type mechanism operates by providing a plurality of second members 114 about the periphery 116 of an aperture 118 formed in a first member 120 which is in contact with a flow of exhaust gas expelled from a patient. The plurality of second members 114 act to block a first portion 118a of aperture 118, thus leaving another portion 118b of aperture 118 unobstructed, thus defining the area of exhaust port 112. It is to be appreciated that second members 118 may be actuated manually or though automated means, similar to the shutter mechanism of a camera, thus making such embodiment as shown in FIGS. 8A and 8B particularly suitable to patient interface systems in which the sizing of exhaust port 112 may be controlled through computerized means.

In addition to the embodiment illustrated, it is to be appreciated that the concepts of the present invention may also be carried out by providing a plurality of apertures and then selectively exposing or covering the entirety or portions of individual apertures to achieve the desired exhaust port sizing (i.e., 1 port, 1-½ ports, 2 ports, etc.).

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1-3. (canceled)

4. An exhaust port assembly for use in a system for delivering a flow of gas from a pressure generating device to the airway of a patient, the exhaust port assembly comprising:
a first member structured to be in communication with the flow of gas; and
a second member movable coupled to the first member wherein the first member and the second member define a cross-sectional area of an exhaust port which is structured to allow the passage therethrough of exhaust gases from the flow of gas, and wherein the second member is moveable among a first position in which the exhaust port has a first cross-sectional area and a second position in which the exhaust port has a second cross-sectional area different than the first cross-sectional area, wherein the first member comprises at least a portion of a first tubular member structured to conduct the flow of gas therethrough and wherein the second member comprises at least a portion of a second tubular member disposed about the first member, and wherein the first tubular member is disposed about a longitudinal axis and wherein the second member is rotatable about, or slidable axially along, the longitudinal axis.

5. The exhaust port assembly of claim 4, wherein the first member comprises an aperture having a length disposed parallel to the longitudinal axis and a width disposed perpendicular to the longitudinal axis, the width varying along the length thereof; wherein the second member is disposed to selectively block a portion of the aperture; and wherein the cross-sectional area of the exhaust port is defined by a portion of the aperture not blocked by the second member.

6. (canceled)

7. The exhaust port assembly of claim 4, wherein the first member comprises a first aperture having a length disposed perpendicular to the longitudinal axis and a width disposed parallel to the longitudinal axis, the width varying along the length thereof; wherein the second member comprises a second aperture having a length disposed along the longitudinal axis, the length being equal to or greater than the width of the first aperture; and wherein the cross-sectional area of the exhaust port is defined by a portion of the first aperture aligned with the second aperture.

8. The exhaust port assembly of claim 4, wherein the first member comprises a first aperture of predetermined cross-sectional area formed therein; wherein the second member comprises a plurality of second apertures of varying cross-sectional areas equal to, or smaller than the cross sectional area of the first aperture, formed therein; wherein the second member is rotatably coupled to the first member in a manner such that each of the second apertures may be selectably aligned with the first aperture; and wherein the cross-sectional area of the exhaust port is defined by the one of the plurality of second apertures aligned with the first aperture.

9. The exhaust port assembly of claim 4, wherein the first member comprises a plurality of first apertures of varying cross-sectional areas formed therein; wherein the second member comprises a second aperture of predetermined cross-sectional area formed therein, the predetermined cross-sectional area of the second aperture being equal to, or larger than any of the cross-sectional areas of the plurality of first apertures; wherein the second member is rotatably coupled to the first member in a manner such that the second aperture may be selectably aligned with each of the plurality of first apertures; and wherein the cross-sectional area of the exhaust port is defined by the one of the plurality of first apertures to which the second aperture is aligned.

10-13. (canceled)

14. A system for delivering a flow of treatment gas to the airway of a patient, the system comprising:
a pressure generating device:
a patient interface
a patient circuit structured to deliver the flow of treatment gas from the pressure generating device to the patient interface; and
an exhaust port assembly comprising;
a first member structured to be in communication with the flow of treatment gas; and
a second member moveably coupled to the first member,
wherein the first member and the second member define a cross-sectional area of an exhaust port which is structured to allow the passage therethrough of exhaust gases from the flow of gas, and wherein the second member is moveable among a first position in which the exhaust port has a first cross-sectional area and a second position in which the exhaust port has a second cross-sectional area different than the first cross-sectional area,
wherein the first member comprises at least a portion of a first tubular member structured to conduct the flow of gas therethrough and wherein the second member comprises at least a portion of a second tubular member disposed about the first member, and
wherein the first tubular member is disposed about a longitudinal axis and wherein the second member is rotatable about, or slidable axially along, the longitudinal axis.

15. The system of claim 14, wherein the first member comprises an aperture having a length disposed parallel to the longitudinal axis and a width disposed perpendicular to the longitudinal axis, the width varying along the length thereof; wherein the second member is disposed to selectively block a portion of the aperture; and wherein the cross-sectional area of the exhaust port is defined by a portion of the aperture not blocked by the second member.

16. (canceled)

17. The system of claim 14, wherein the first member comprises a first aperture having a length disposed perpendicular to the longitudinal axis and a width disposed parallel to the longitudinal axis, the width varying along the length thereof; wherein the second member comprises a second aperture having a length disposed along the longitudinal axis, the length being equal to or greater than the width of the first aperture; and wherein the cross-sectional area of the exhaust port is defined by a portion of the first aperture aligned with the second aperture.

18. The system of claim 14, wherein the first member comprises a first aperture of predetermined cross-sectional area formed therein; wherein the second member comprises a plurality of second apertures of varying cross-sectional areas equal to, or smaller than the cross sectional area of the first aperture, formed therein; wherein the second member is rotatably coupled to the first member in a manner such that each of the second apertures may be selectably aligned with the first aperture; and wherein the cross-sectional area of the exhaust port is defined by the one of the plurality of second apertures aligned with the first aperture.

19. The system of claim 14, wherein the first member comprises a plurality of first apertures of varying cross-sectional areas formed therein; wherein the second member comprises a second aperture of predetermined cross-sectional area formed therein, the predetermined cross-sectional area of the second aperture being equal to, or larger than any of the cross-sectional areas of the plurality of first apertures; wherein the second member is rotatably coupled to the first member in a manner such that the second aperture may be selectably aligned with each of the plurality of first apertures; and wherein the cross-sectional area of the exhaust port is defined by the one of the plurality of first apertures to which the second aperture is aligned.

20. (canceled)
